(19) United States
(12) Patent Application Publication
(10) Pub. No.: US 2005/0043689 A1
(43) Pub. Date: Feb. 24, 2005

(54) Sealing piston for syringe

(75) Inventor: Chin-Rong Chen, Taipei (TW)

Correspondence Address: BACON & THOMAS, PLLC, 625 SLATERS LANE, FOURTH FLOOR, ALEXANDRIA, VA, 22314, US

(73) Assignee: LITE-ON IT CORPORATION, Taipei (TW)

(21) Appl. No.: 10/917,291

(22) Filed: Aug. 13, 2004

(30) Foreign Application Priority Data
Aug. 19, 2003 (TW) 92215037

(51) Int. Cl. A61M 5/315

(52) U.S. Cl. 604/218

(57) ABSTRACT

The present invention is directed to a sealing piston for syringe. The sealing piston includes a first sealing element, a second sealing element, and a third sealing element. The second sealing element is elastic and expandable. One end surface of the second sealing element is connected with the first sealing element, and another end surface of the second sealing element is connected with the third sealing element for constraining the second sealing element. The present invention not only effectively resolves the air-leakage problem of a conventional syringe, but also enhances dispensing accuracy.

This application claims the benefit of Taiwan application Serial No. 92215037, filed Aug. 19, 2003, the subject matter of which is incorporated herein by reference.

### BACKGROUND OF THE INVENTION

1. Field of the Invention

The invention relates in general to a sealing piston, and more particularly to a sealing piston applied in a syringe.

2. Description of the Related Art

The syringe has a wide range of application, ranging from an ordinary syringe for spraying a thread-like topping on a cake, a syringe medical for ejecting medicine, to an industrial syringe for ejecting a fixed amount of glue or oil and so on. Generally speaking, the syringe has a cylinder and a piston, wherein the cylinder is for containing an ejection substance, while the piston is loaded into the cylinder and pushes the ejection substance contained in the cylinder to be ejected by means of an external force.

A conventional industrial syringe is shown in FIG. 1. A piston 2A is loaded into a cylinder 1A, wherein the piston 2A, which is made of an elastic material such as rubber, has an outer diameter wider than the inner diameter of the cylinder 1A. When the piston 2A is loaded into the cylinder 1A, the outer wall of the piston 2A will be squeezed to produce a sealing effect between the cylinder 1A and the piston 2A; furthermore, by applying an external force on the piston 2A, the substance contained in the cylinder 1A will thus be ejected.

However, the syringe for industrial purpose has cylinder distortion problem caused by ejection pressure and air-leakage problem. When the cylinder is distorted by the ejection pressure, the sealing effect between the cylinder and the piston will become worse. Then, the air used to provide a pressure to push the piston may penetrate into the ejection substance contained in the tube because of the worse sealing effect, and the dispensing accuracy will deteriorate, the ejection substance might be blocked and cannot be ejected and, worst of all, the ejection substance may even ooze out of the cylinder and spread on the machine, causing damage to the machine. An invention to resolve the above problems is needed.

### SUMMARY OF THE INVENTION

It is therefore an object of the invention to provide a sealing piston applied in a syringe, the sealing piston in the present invention can effectively resolve the leakage problem of a conventional syringe and enhance the dispensing accuracy of the syringe.

The invention achieves the above-identified object by providing a sealing piston applied in a syringe. The sealing piston includes a first sealing element, a second sealing element, and a third sealing element. The second sealing element is elastic and expandable. One end surface of the second sealing element is connected with the first sealing element, and another end surface of the second sealing element is connected with the third sealing element for constraining the second sealing element.

Other objects, features, and advantages of the invention will become apparent from the following detailed description of the preferred but non-limiting embodiments. The following description is made with reference to the accompanying drawings.

### BRIEF DESCRIPTION OF THE DRAWINGS

FIG. 1 (Prior Art) is a structural diagram of a conventional industrial syringe.

FIG. 2 is a structural diagram of the sealing piston according to the invention.

FIG. 3 is an exploded view of the sealing piston according to the invention.

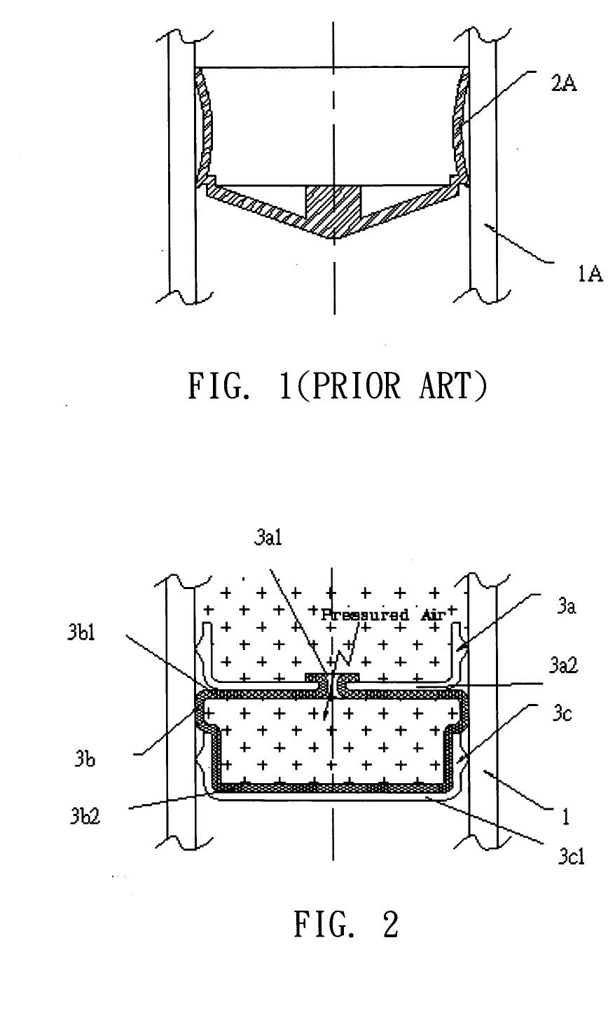

### DETAILED DESCRIPTION OF THE INVENTION

The preferred embodiment of the invention disclosed below relates to an industrial purpose syringe for dispensing glue or oil. Nonetheless, the invention can be applied to all of syringe related products.

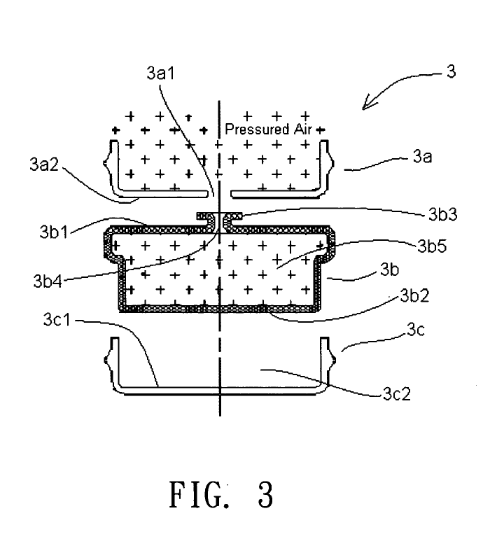

Referring to FIG. 2 and FIG. 3, the structural diagrams of a sealing piston according to the invention are shown. The structural diagram in FIG. 2 includes a cylinder 1 and a sealing piston 3. The cylinder 1 has a first aperture and a second aperture (not shown here), wherein the outer diameter of the first aperture is larger than the outer diameter of the second aperture. The sealing piston 3 includes a first sealing element 3a, a second sealing element 3b and a third sealing element 3c. As shown in FIG. 3, the first sealing element 3a, which has a central hole 3a1, is made of elastic material such as rubber, plastics or an equivalent material thereof. In the preferred embodiment, the first sealing element 3a is a lip seal ring, and the outer diameter of the first sealing element 3a is larger than the inner diameter of the cylinder 1 to produce the sealing effect between the first sealing element 3a and the cylinder 1. The second sealing element 3b, which is made of an elastic material such as the Teflon or an equivalent material thereof, has an inner vacancy 3b5 and a protrusive portion 3b3 wherein the protrusive portion 3b3 can pass though the central hole 3a1 and fixed on the central hole 3a1, so that an end surface 3b1 of the second sealing element 3b can be connected with an end surface 3a2 of the first sealing element 3a. Besides, the protrusive portion 3b3 has a channel 3b4 where the pressured air used to push the sealing piston 3 can pass through into the inner vacancy 3b5 from the first sealing element 3a. The second sealing element 3b is softer than the first sealing element 3a and expands when an external pressure, such as pressured air, is filled in the inner vacancy 3b5 of the second sealing element 3b. The third sealing element 3c is made of an elastic material such as rubber, plastics or an equivalent material thereof. Another end surface 3b2 of the second sealing element 3b is contained in the inner space 3c2 surrounded by the inner surface 3c1 of the third sealing element 3c, so that the inner surface 3c1 of the third sealing element 3c is connected with another end surface 3b2 of the second sealing element 3b. In the preferred embodiment, the third sealing element 3c is a lip seal ring, and the outer diameter of the third sealing element 3c is larger than the inner diameter of the cylinder 1 to produce the sealing effect between the third sealing element 3c and the cylinder 1.

When the sealing piston 3 is loaded into the cylinder 1 via the first aperture of the cylinder 1, the first sealing element 3a and the third sealing element 3c will form a sealing effect with the inner wall of the cylinder 1 because the outer diameter of the first sealing element 3a and that of the third sealing element 3c are both larger than the inner diameter of the cylinder 1. When an air pressure is applied onto the sealing piston 3 via the first aperture of the cylinder 1 to push the sealing piston 3, the pressured air, having filled in the first sealing element 3a, will enter into the inner vacancy 3b5 of the second sealing element 3b via the channel 3b4. Having been filled with the pressured air, the second sealing element 3b will expand and match tightly with the inner wall of the cylinder 1 to produce an even better airtight effect. While the inner vacancy 3b5 of the second sealing element 3b is filled with the pressured air, the second sealing element 3b is shaped and constrained by the inner surface 3c1 of the third sealing element 3c. Then, the pressured air will start to push the sealing piston 3. Therefore, the ejection substance contained in the cylinder 1 will be ejected via the second aperture of the cylinder 1. By doing so, even the inner wall of the cylinder 1 is slightly distorted by the air pressure, the second sealing element 3b will resume expanding and matching tightly with the distorted inner wall of the cylinder 1. Therefore, the invention not only effectively resolves the air-leakage problem, which occurs when air penetrates into the ejection substance contained in the tube, and prevents ejection blockage, which causes the ejection substance to be blocked or even ooze out of the cylinder, spread on the machine and cause damage, but also enhances dispensing accuracy.

While the invention has been described by way of example and in terms of a preferred embodiment, it is to be understood that the invention is not limited thereto. On the contrary, it is intended to cover various modifications and similar arrangements and procedures, and the scope of the appended claims therefore should be accorded the broadest interpretation so as to encompass all such modifications and similar arrangements and procedures.

1. A sealing piston for syringe, comprising:
a first sealing element;
a second sealing element, which is elastic and expandable and one end surface of which is connected with the first sealing element; and
a third sealing element, connected with another end surface of the second sealing element.

2. The sealing piston according to claim 1, wherein the first sealing element has a central hole.

3. The sealing piston according to claim 2, wherein the second sealing element has a protrusive portion, and the first sealing element and the second sealing element are connected by the central hole and the protrusive portion.

4. The sealing piston according to claim 1, wherein the second sealing element has an inner vacancy and a channel, and the channel communicates the inner vacancy and the first sealing element.

5. The sealing piston according to claim 1, wherein the second sealing element, which is a soft material with elasticity, expands when an external pressure is applied thereon.

6. The sealing piston according to claim 1, wherein the first sealing element is made of rubber, or plastics.

7. The sealing piston according to claim 1, wherein the first sealing element is a lip seal ring.

8. The sealing piston according to claim 1, wherein the second sealing element is made of Teflon.

9. The sealing piston according to claim 1, wherein the third sealing element is made of rubber, or plastics.

10. The sealing piston according to claim 1, wherein the third sealing element is a lip seal ring.
